(19) United States
(12) Patent Application Publication
(10) Pub. No.: US 2019/0021351 A1
(43) Pub. Date: Jan. 24, 2019

(54) FLAVOURED MILK PRODUCT

(71) Applicant: NESTEC S.A., Vevey (CH)

(72) Inventor: Marcel Braun, Konolfingen (CH)

(21) Appl. No.: 16/064,062

(22) PCT Filed: Dec. 20, 2016
(86) PCT No.: PCT/EP2016/082029

§ 371 Date: Jun. 20, 2018

(30) Foreign Application Priority Data
Dec. 22, 2015 (EP) 15202070.7

(51) Int. Cl. A23C 9/123; A23C 9/13; A23C 9/12; A23G 1/56; C12R 1/225; C12P 17/12

(52) U.S. Cl. CPC A23C 9/123; A23C 9/1322; C12P 17/12; A23G 1/56; C12R 1/225; A23C 9/1216

(57) ABSTRACT

The invention provides a method for producing a flavoured milk product, the method comprising the steps of: (a) providing milk source; (b) fermenting the milk source using at least one microorganism, to produce a fermented milk source having an acidic pH; and (c) increasing the pH of the fermented milk source using ammonia, wherein the ammonia reacts to produce 2,3,5,6-tetramethyl pyrazine.

### FIELD OF INVENTION

The present invention relates to methods for producing flavoured milk products. In particular, the invention relates to methods for producing flavoured milk products with a chocolate flavour. The generation of flavour in milk-based products is achieved using microorganisms during fermentation of a milk source. The method of the invention as described herein enables the production of a chocolate flavour in milk products without the need to add chocolate or cocoa products.

### BACKGROUND

Fermented milk products are major consumer products. Fermented milk products can be, for example, cheeses, buttermilks and yoghurts. Fermented milk products are manufactured by fermenting milk.

Fermentation is the conversion of carbohydrates to organic acids or other compounds using bacteria strains.

Milk contains the carbohydrate lactose. During fermentation of milk, microorganisms such as bacteria ferment the carbohydrate lactose to produce lactic acid. The production of lactic acid results in an acidification of the milk during the manufacture of the fermented milk product. During fermentation of the milk, other reactions may occur between other substances present in the milk and the microorganisms. These reactions can produce many different compounds associated with different flavours and aromas.

The microorganisms used to ferment the milk can be lactic acid bacteria strains such as Lactobacillus, Leuconostoc, Pediococcus, Lactococcus and Streptococcus, as well as the more peripheral Aerococcus, Carnobacterium, Enterococcus, Oenococcus, Sporolactobacillus, Teragenococcus, Vagococcus and Weisella. These lactic acid bacteria strains belong to the order Lactobacillales.

Fermented milk products have a wide variety of flavours and aromas depending upon the milk source used and the microorganisms used to ferment the milk source.

Consumers have preferences for milk and related products with particular flavours and/or aromas, such as a chocolate flavour or aroma. Consumers also have a preference for food products having flavours and/or aromas that are naturally produced without the need for the addition of artificial (i.e. synthetic) flavour- or aroma-associated compounds, which are often negatively perceived by the consumer.

Chocolate flavour and/or aroma can be added to a milk product or other food product through the addition of cocoa powder. However, this results in a brown colouration of the product, which can be unwanted. As an alternative, specific flavourings can be added to the product; however, these are often artificial in nature and require the product to be labelled as containing non-natural ingredients.

Therefore, there is a need to provide alternative and/or improved methods for producing flavoured milk products with specific flavours and/or aromas.

WO 2008/049581 describes a method for promoting natural tastes and flavours in milk-based products.

WO 2012/085009 describes the fermentation of a milk source with a Lactobacillus lactis to form a fermented milk product with a flavour and aroma.

### SUMMARY OF INVENTION

The present invention addresses the above prior art problems by providing a method for producing a flavoured milk product, together with flavoured milk products, as specified in the claims.

In one aspect, there is provided a process for producing a flavoured milk product comprising the steps of: (a) providing a milk source; (b) fermenting the milk source using at least one microorganism, to produce a fermented milk source having an acidic pH; and (c) increasing the pH of the fermented milk source using ammonia, wherein the ammonia reacts with the fermented milk source to produce 2,3,5,6-tetramethyl pyrazine, thereby producing a flavoured milk product.

In one embodiment, the ammonia in step (c) is produced by adding urease to the fermented milk source, and/or step (c) comprises adding ammonia or an ammonium salt to the fermented milk source.

In one embodiment, the pH in step (c) is increased to about 6 to about 8.0, preferably about 6.25 to about 7.7, more preferably about 6.5 to about 7.5 and most preferably about 6.7 to about 7.3.

In one embodiment, step (c) further comprises subjecting the fermented milk source to a heat treatment, preferably wherein the heat treatment comprises at least one of: Ultra High Temperature (UHT) processing, spray drying, roller drying, vacuum belt drying, continuous drying or batch drying, and preferably UHT processing or spray drying.

In one embodiment, the heat treatment comprises UHT treatment which is preferably conducted at a temperature of greater than about 130° C., preferably about 135-155° C., more preferably about 140-150° C., and most preferably about 145-148° C.

The UHT treatment may be carried out over a period of less than about 10 seconds, preferably about 1-7 seconds, particularly about 2-6 seconds, more preferably about 2-5 seconds, and most preferably about 2-4 seconds.

In one embodiment, the at least one microorganism is a lactic acid bacterium (Lactobacillales), preferably selected from the group consisting of: Lactobacillus, Leuconostoc, Pediococcus, Lactococcus, Streptococcus, Aerococcus, Carnobacterium, Enterococcus, Oenococcus, Sporolactobacillus, Tetragenococcus, Vagococcus, and Weissella, more preferably selected from the group consisting of Lactobacillus, Lactococcus, and Streptococcus, and most preferably selected from the group Lactobacillus and Lactococcus.

In one embodiment, the at least one microorganism is a Lactococcus bacterium, preferably Lactococcus lactis, more preferably Lactococcus lactis subsp. Lactis, and most preferably Lactococcus lactis subsp. Lactis biovar. Diacetylactis.

In one embodiment, the lactic acid bacterium is selected from the bacteria deposited at the Collection Nationale de Cultures de Microorganismes under the following accession numbers: CNCM I-1962, CNCM I-2116, CNCM I-4404 and CNCM I-4405; preferably CNCM I-1962, CNCM I-4404 and CNCM I-4405.

In one embodiment, the 2,3,5,6-tetramethyl pyrazine is present at a concentration of at least about 1 parts-per-million (ppm), preferably at least about 5 ppm, more preferably at least about 10 ppm, most preferably at least about 25 ppm, and particularly at least about 50 ppm.

In one embodiment, 2,3,5,6-tetramethyl pyrazine is present at a concentration of about 1 to about 1000 ppm, preferably about 5 to about 500 ppm, more preferably about 10 to about 250 ppm, most preferably about 20 to about 150 ppm, and particularly about 50 to about 100 ppm.

In one embodiment, the milk source in step (a) is an amino acid supplemented milk source.

In one embodiment, the milk source in step (a) is an amino acid supplemented milk source comprising one or more amino acids selected from the group consisting of: L-phenylalanine, L-leucine, L-isoleucine and L-valine.

In one embodiment, the amino acid supplemented milk source comprises at least L-phenylalanine, or at least a combination of L-phenylalanine and L-leucine.

In one embodiment, the amino acid supplemented milk source is fermented using the bacterium deposited at the Collection Nationale de Cultures de Microorganismes under the accession number CNCM I-1962.

In one embodiment, the flavoured milk product further comprises 5-methyl-2-phenyl-2Z-hexenal, preferably in an amount of: greater than 1 ppm, about 1-1000 ppm, about 5-500 ppm, about 10-200 ppm, about 10-100 ppm, about 20 to about 100 ppm or about 20-50 ppm.

The amino acid supplemented milk source may be fermented using the bacteria deposited at the Institut Pasteur Collection Nationale de Cultures de Microorganismes under the following accession numbers: CNCM I-1962 and CNCM I-2116, together or sequentially.

The flavoured milk product may further comprise 2-phenylbutenal, preferably in an amount of greater than 0.1 ppm, about 0.2-100 ppm, about 0.5-50 ppm, about 1-50 ppm, about 1-20 ppm, or about 10-20 ppm.

In one embodiment, the amino acid supplemented milk source in step (a) is prepared by hydrolysis of milk proteins in a milk source, preferably by adding at least one protease to a milk source, thereby providing an amino acid supplemented milk source.

In one embodiment, the at least one amino acid is selected from the group consisting of: L-phenylalanine, L-leucine, L-isoleucine and L-valine.

In one embodiment, the amino acid supplemented milk source comprises at least L-phenylalanine, or at least a combination of L-phenylalanine and L-leucine.

In one embodiment, the at least one protease is an endoprotease and/or an exopeptidase, preferably a fungal protease/peptidase complex.

The at least one protease may be derived from Aspergillus oryzae, preferably A. oryzae strain ATCC 42149/RIB 40 (yellow koji mould).

In one embodiment, the amino acid supplemented milk source in step (a) is prepared by adding at least one amino acid to a milk source to provide an amino acid supplemented milk source.

The at least one amino acid may be selected from the group consisting of: L-phenylalanine, L-leucine, L-isoleucine and L-valine.

In one embodiment, the amino acid supplemented milk source comprises at least L-phenylalanine, or at least a combination of L-phenylalanine and L-leucine.

The process may further comprise drying and/or homogenising the flavoured milk product.

In another aspect, there is provided a process for preparing a food product containing a flavoured milk product, comprising: (a) preparing a flavoured milk product by a process as described above, and (b) adding the flavoured milk product to a food to form a food product.

In a further aspect, there is provided a process for flavouring a food product comprising: (a) preparing a flavoured milk product by a process as described above, and (b) adding the flavoured milk product to a food product.

In yet a further aspect, there is provided a flavoured milk product comprising 2,3,5,6-tetramethyl pyrazine at a concentration of at least about 10 parts-per-million (ppm), preferably at least about 20 ppm, more preferably at least about 30 ppm, most preferably at least about 40 ppm, and particularly at least about 50 ppm. In one embodiment, said flavoured milk product is free of cocoa and chocolate.

The 2,3,5,6-tetramethyl pyrazine may be present at a concentration of about 10 to about 1000 ppm, preferably about 20 to about 500 ppm, more preferably about 30 to about 250 ppm, most preferably about 40 to about 150 ppm, and particularly about 50 to about 100 ppm.

The flavoured milk product may further comprise 5-methyl-2-phenyl-2Z-hexenal, preferably in an amount of greater than about 5 ppm, about 1-1000 ppm, about 5-500 ppm, about 10-200 ppm, about 10-100 ppm, about 20 to about 100 ppm or about 20-50 ppm.

The flavoured milk product may further comprise 2-phenylbutenal, preferably in an amount of about greater than about 5 ppm, about 10-1000 ppm, about 20-500 ppm, about 20-200 ppm, or about 50-200 ppm.

In yet a further aspect, there is provided a food product containing a flavoured milk product as described above.

In yet a further aspect, there is provided a flavoured milk product or a food product comprising 2,3,5,6-tetramethyl pyrazine, which is obtainable or obtained by the process as described above.

In yet a further aspect, 2,3,5,6-tetramethyl pyrazine is present at a concentration of at least about 10 parts-per-million (ppm), preferably at least about 20 ppm, more preferably at least about 30 ppm, most preferably at least about 40 ppm, and particularly at least about 50 ppm in the flavoured milk product or the food product obtainable or obtained by the process of the present invention.

In one embodiment, 2,3,5,6-tetramethyl pyrazine is present at a concentration of about 10 to about 1000 ppm, preferably about 20 to about 500 ppm, more preferably about 30 to about 250 ppm, most preferably about 40 to about 150 ppm, and particularly about 50 to about 100 ppm.

In one embodiment, the flavoured milk product obtainable or obtained by the process as described above further comprises 5-methyl-2-phenyl-2Z-hexenal, preferably in an amount of about greater than about 5 ppm, about 1-1000 ppm, about 5-500 ppm, about 10-200 ppm, about 10-100 ppm, about 20 to about 100 ppm or about 20-50 ppm.

In one embodiment, the flavoured milk product obtainable or obtained by the process as described above further comprises 2-phenylbutenal, preferably in an amount of greater than about 5 ppm, about 10-1000 ppm, about 20-500 ppm, about 20-200 ppm, or about 50-200 ppm.

In one embodiment, the flavoured milk product as described above or the food product as described above is in the form of a liquid or a solid, preferably a solid, and more preferably a powder.

In yet a further aspect, there is provided the use of ammonia or an ammonium salt for enhancing the flavour of a fermented milk product, preferably by increasing the concentration of 2,3,5,6-tetramethyl pyrazine in the fermented milk product. Preferably, the flavour is a chocolate flavour.

In an embodiment, there is provided the use of ammonia or an ammonium salt for conferring a chocolate flavour and/or for enhancing a chocolate flavour in a fermented milk product, preferably by increasing the concentration of 2,3,5,6-tetramethyl pyrazine in the fermented milk product.

In yet a further aspect, there is provided the use of a urease for enhancing the flavour of a milk product, preferably by increasing the concentration of 2,3,5,6-tetramethyl pyrazine in a fermented milk product. Preferably, the flavour is a chocolate flavour.

In an embodiment, there is provided the use of a urease for conferring a chocolate flavour and/or for enhancing a chocolate flavour in a fermented milk product, preferably by increasing the concentration of 2,3,5,6-tetramethyl pyrazine in the fermented milk product.

### DESCRIPTION OF FIGURES

FIG. 1 shows chemical pathways related to the generation of 2,3,5,6-tetramethyl pyrazine.

FIG. 2 shows the generation of 5-methyl-2-phenyl-2Z-hexenal from phenyl acetaldehyde and 3-methylbutanal, catalysed by aldolase.

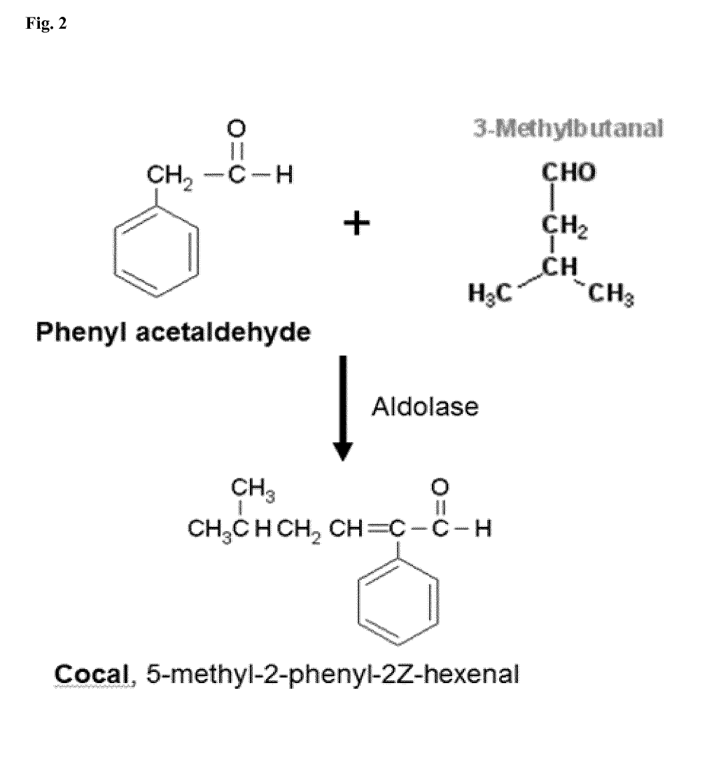

FIG. 3 shows the generation of 2-phenylbutenal from phenyl acetaldehyde and acetaldehyde, catalysed by aldolase.

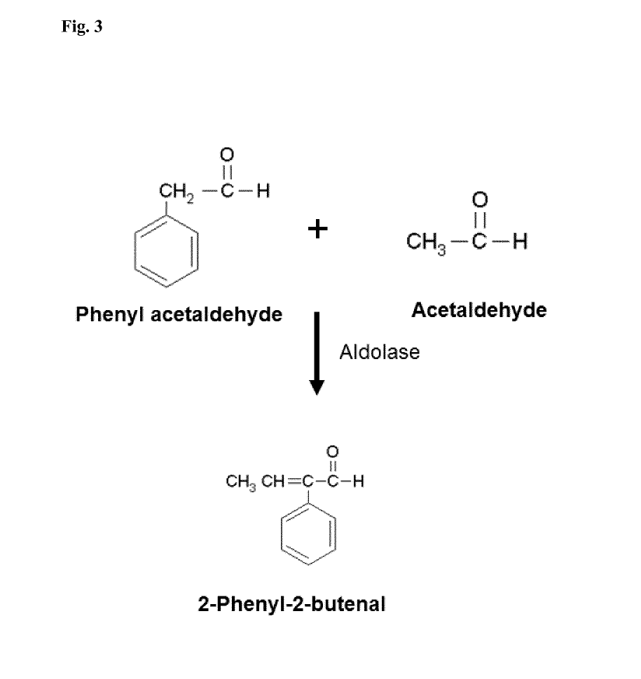

FIG. 4 shows the composition of two flavoured milk products following drying for different durations.

FIG. 5 shows the concentration of 2,3,5,6-tetramethyl pyrazine obtained using ammonia neutralisation and potassium hydroxide neutralisation followed by spray drying.

FIG. 6 shows the evolution of the concentration of 5-methyl-2-phenyl-2Z-hexenal obtained by fermentation of a milk product.

### DETAILED DESCRIPTION OF THE INVENTION

The present invention provides a method for producing a flavoured milk product, the method comprising the steps of: (a) providing a milk source; (b) fermenting the milk source using at least one microorganism, to produce a fermented milk source having an acidic pH; and (c) increasing the pH of the fermented milk source using ammonia, wherein the ammonia reacts with the fermented milk source to produce 2,3,5,6-tetramethyl pyrazine, thereby producing a flavoured milk product.

The present inventors have found that the use of ammonia to increase the pH of the fermented milk source leads to the improved production in the milk source of the compound 2,3,5,6-tetramethyl pyrazine, which is associated with a chocolate flavour and/or aroma, and in particular a rich or dark chocolate flavour and/or aroma. Thus, the method of the invention advantageously allows for the production of a flavoured milk product having a chocolate flavour and/or aroma, without the need for the addition of artificial flavourings or cocoa powder.

Thus, in one embodiment, a flavoured milk product produced according to the method of the invention does not contain cocoa powder or chocolate.

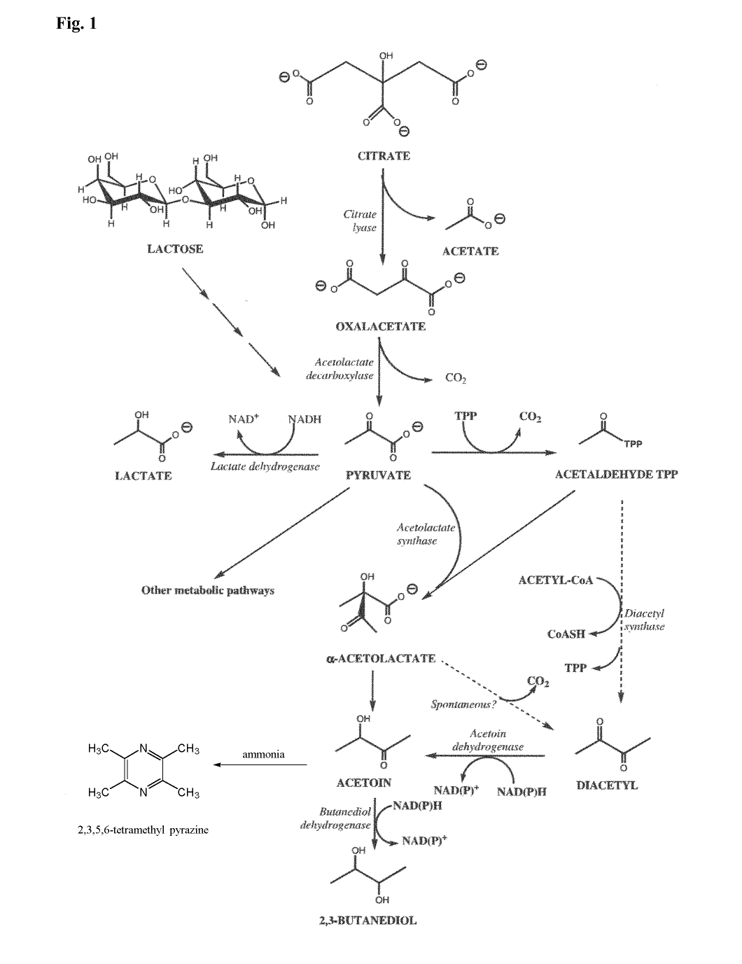

Without wishing to be bound by theory, it is believed that 2,3,5,6-tetramethyl pyrazine is produced when the ammonia reacts with the compound acetoin in the fermented milk source (FIG. 1). Acetoin is produced via a number of chemical pathways catalysed by enzymes present in the microorganisms used to ferment the milk source (see: McSweeney P, International Journal of Dairy Technology, (2004) Vol 57, No 2/3, 127-144).

The fermentation process increases the acidity of the milk product, lowering its pH. Following fermentation, the pH of the fermented milk product may be between about 4.5 and about 5.8; for example, between about 5.0 and about 5.8, or between about 5.4 and about 5.7.

As used herein, “increasing the pH of the fermented milk” means that the acidity of the fermented milk is reduced. The pH is increased to a value of about 6.0 to about 8.0; for example about 6.2 to about 7.7, about 6.5 to about 7.5, or about 6.7 to about 7.3. In one embodiment, the pH is increased to a pH value between 6 and 7 (for example between 6.0 and 7.0, between 6.2 and 7.0, between 6.4 and 7.0, or between 6.6 and 7.0; or about 6.1, 6.2, 6.3, 6.4, 6.5, 6.6, 6.7, 6.8 or 6.9).

In a preferred embodiment, the pH value is measured at room temperature, for example between about 20 and about 30° C. (e.g. about 20 to about 25° C.).

Preferably the fermented milk is neutralised—i.e. its pH is adjusted to approximately 7 (for example, about 6.7 to about 7.3, preferably about 6.8 to about 7.2, more preferably about 6.9 and about 7.1, more preferably about 7.0). Thus, in one embodiment, the fermented milk is neutralised using ammonia.

To increase the pH of the fermented milk source using ammonia. The ammonia may be added directly to the fermented milk source or may be generated in the fermented milk source in situ, for example by the action of an enzyme. In one embodiment, the ammonia is added to the milk source in the form of ammonia (NH₃), which may be liquid ammonia or aqueous ammonia (also known as ammonium hydroxide, NH₄OH, which contains the ammonium ion NH₄⁺). The aqueous ammonia may be a 10-28% (e.g. 10, 15, 20, 25, or 28%) solution of NH₃ in water. In one embodiment, ammonia is added to the milk in the form of an ammonia source, such as an ammonium salt (containing the ion NH₄⁺), for example ammonium carbonate (E503) or ammonium chloride. The amount of ammonia solution (or solution of an ammonia source) added to the fermented milk source may range from about 4 to about 40 grams per litre depending on the initial pH following fermentation and the total milk solids respective to the amount of formed organic acids and the buffer capacity of the fermented milk. The amount of ammonia used may be calculated based on the amount required to neutralise the lactic acid produced during fermentation.

The ammonia may also be generated in the fermented milk source through the action of an enzyme. A suitable enzyme is urease, which catalyses the hydrolysis of urea into carbon dioxide and ammonia in the following reaction: (NH₂)₂CO+H₂O→CO₂+2NH₃. When used in the method of the invention, urease hydrolyses urea that is present naturally in the milk product.

Urease preparations are commercially available, for example urease obtained from the Jack bean (Canavalia ensiformis). Urease functions at a range of pH values, with an optimum range of pH 6.5 to 7.5. Urease may be added to the fermented milk source at a concentration of about 350 to about 450 U/kg (for example, about 400 U/kg) milk concentrate, which naturally contains about 1 g urea/kg milk concentrate. The urease may be incubated for about 20 to about 40 minutes (for example, about 20, about 25, about 30, about 25, or about 40 minutes) at a temperature of about 20 to about 30° C. (for example, about 20, about 25 or about 30° C.).

Optionally, when urease is employed, the pH of the fermented milk source may be increased to achieve the preferred pH ranges as set above by the addition of a base, for example potassium hydroxide, sodium hydroxide, ammonia or an ammonia source. Preferably, when using urease, the pH of the fermented milk source may be increased by both the addition of ammonia and the use of urease enzyme, either sequentially or in combination. The pH may be increased to the optimum range of 6.5 to 7.5 (for example, using potassium hydroxide or sodium hydroxide) prior to the addition of urease.

Thus, in one embodiment, the ammonia is produced in the fermented milk source by adding urease to the fermented milk source. The use of urease in this manner avoids the need to add exogenous ammonia or an ammonium salt to the fermented milk product. This can be advantageous due to food regulatory or labelling provisions that would apply to the use of exogenous ammonia or an ammonium salt.

The milk source used in the method of the invention can be any type of milk, such as cow milk, sheep milk, goat milk, buffalo milk or any mixtures thereof. The milk source may be UHT-treated milk, pasteurised milk, or non-pasteurised milk. The milk source may be full fat milk, skimmed milk or semi-skimmed milk. Furthermore, the milk source may be a fresh milk, recombined milk, or milk containing vegetable fat or any mixtures thereof.

Fermentation of the milk source by the at least one microorganism is required for the generation of flavour- and/or aroma-associated compounds.

Examples of flavour- and/or aroma-associated compounds that may be produced during a fermentation process include 3-methyl-butanal, 2-methyl-butanal, 2-methyl-1-propanal, 2-phenylacetaldehyde, 2, 3-butandione (diacetyl), 3-hydroxy-2-butaneone (acetoin), 2-phenyl-2-butenal and other phenyl-aldehydes (C10-16), benzaldehyde, 5-methyl-2-phenyl-cis-2-hexenal (cocal), 2,4,5-trimethyl oxazole, delta-octalactone (5-octanolide), delta-decalactone (5-decanolide), delta-dodecalactone (5-dodecanolide), butanoic acid, hexanoic acid, octanoic acid, decanoic acid, dodecanoic acid, 4-hydroxy-2,5-dimethyl-3 (2H)-furanone (furaneol). By phenyl-aldehydes (C10-16) is meant phenyl-aldehydes having a carbon chain of 10 to 16 carbon atoms.

Certain flavour- and/or aroma-associated compounds may be derived ultimately from amino acids metabolised by the at least one microorganism. In certain embodiments, the milk source may be supplemented with amino acids. In the present context, supplementation with amino acids means that additional amino acids are provided in the milk source in addition to any amino acids that are naturally present. Reference to naturally present amino acids means free amino acids only, and does not include amino acid residues that are part of polypeptides such as proteins.

Supplementation of a milk source with amino acids can be achieved either through directly adding amino acids to the milk source or through proteolytic digestion of milk source proteins to increase the concentration of free amino acids in the milk source. In order to supplement the milk source at least one of an amino acid, a protease (or peptidase) or any mixture thereof may be added to the milk source.

An amino acid supplemented milk source may be prepared by adding at least one amino acid to a milk source. The at least one amino acid may be L-phenylalanine, L-leucine, L-isoleucine, and/or L-valine. Preferably the at least one amino acid is selected from L-phenylalanine and/or L-leucine, as these amino acids are particularly advantageous precursors of 5-methyl-2-phenyl-2Z-hexenal and 2-phenyl-2-butenal.

An amino acid supplemented milk source may also be prepared by adding at least one protease to a milk source. The protease hydrolyses proteins naturally present in the milk source to produce free amino acids, thus supplementing the milk source with amino acids. The free amino acids produced by the action of the protease may include L-phenylalanine, L-leucine, L-isoleucine, or L-valine.

The protease may be an exoprotease, one which cleaves the terminal or penultimate peptide bond of a polypeptide, thus releasing free amino acids from a terminus of a polypeptide. Alternatively, the protease may be an endoprotease, one which cleaves a non-terminal peptide bond (i.e. one within a polypeptide chain rather than at its end).

Supplementation of a milk source with amino acids may be achieved by adding to the milk source a mixture comprising at least one exoprotease and at least one endoprotease.

The at least one protease may be derived from a microorganism, for example Aspergillus oryzae, Aspergillus sojae, Rhizophus oryzae, Bacillus subtilis, L. helveticus, S. thermophiles, L. plantarum, or L. lactis subspecies, or from plants, such as Ananas comosus. The Aspergillus oryzae strain may be ATCC 42149/RIB40 (yellow koji mould).

The at least one protease may be a fungal protease/peptidase complex.

The at least one protease may be derived from Aspergillus oryzae.

The at least one protease may be an enzyme preparation derived from Aspergillus oryzae, for example an enzyme preparation which contains both endoprotease and exopeptidase activities. An example of such an enzyme preparation derived from Aspergillus oryzae is Flavourzyme™, which is produced by the Novo Nordisk company. Flavourzyme™ is a fungal protease/peptidase complex that is suitable for hydrolysis of proteins under neutral or acidic conditions, and which contains both endoprotease and exopeptidase activities.

The at least one protease (or peptidase) can be added with or without the at least one amino acid. The at least one of the amino acid, the protease (or peptidase) or any mixture thereof are added to the milk source in an amount of 0.01 to 5 wt. %, preferably 0.001 to 2 wt. %, more preferably in an amount of 0.03 to 1.0 wt. %, most preferably 0.05 to 0.3 wt. %.

The amino acid supplemented milk source is fermented using at least one microorganism added to the supplemented milk source, to produce a fermented milk source having an acidic pH. Following fermentation, the pH of the fermented milk product may be between about 4.5 and about 5.8; for example, between about 5.0 and about 5.8, or between about 5.4 and about 5.7.

As part of the fermentation process, the amino acids react to provide direct or indirect conversion products responsible for particular flavours.

In one embodiment, the amino acid supplemented milk source is fermented using at least one microorganism selected from a lactic acid bacterium (Lactobacillales). Lactic acid bacteria are a group of Gram-positive, acid-tolerant, rod- or cocci-shaped bacteria sharing common metabolic characteristics. Lactic acid bacteria produce lactic acid as a major end product of carbohydrate fermentation.

The present disclosure describes lactic acid bacteria deposited at the Institut Pasteur Collection Nationale de Cultures de Microorganismes, Paris, France (CNCM) under the following accession numbers: CNCM I-1962, CNCM I-2116, CNCM I-4404, CNCM I-4405.

CNCM I-1962 CNCM I-4404, and CNCM I-4405 were deposited by NESTEC SA, Avenue Nestlé 55, CH-1800 Vevey, Switzerland. CNCM I-2116 was deposited by SOCIETE DES PRODUITS NESTLE SA, Avenue Nestlé 55, CH-1800 Vevey, Switzerland.

Bacterium CNCM I-1962 is described in WO 2012/085009 and WO 2012/085010, the contents of which are herein incorporated by reference. Bacteria CNCM I-4404 and CNCM I-4405 are described in WO 2012/085010.

Methods for producing fermented milk products using bacteria including CNCM I-1962 are described in WO 2012/085009.

The present inventors have found that particularly advantageous results are obtained when fermenting an amino acid supplemented milk source using specific strains of lactic acid bacteria. Thus, in one embodiment, the lactic acid bacterium is a Lactococcus lactis subspecies (subsp.) lactis bacterium. In one embodiment, the lactic acid bacterium is a Lactococcus lactis subsp. lactis biovar diacetylactis bacterium. In one embodiment, the lactic acid bacterium is selected from the bacteria deposited at CNCM under the following accession numbers: CNCM 1-1962, CNCM I-2116, CNCM I-4404 and CNCM I-4405; preferably CNCM I-1962, CNCM 1-4404 and CNCM I-4405. The present inventors have found that the use of each of these bacteria advantageously increases the production of 5-methyl-2-phenyl-2Z-hexenal and/or 2-phenylbutenal.

The supplemented milk source with at least one microorganism added is preferably fermented for between 6 and 24 hours (for example, 6, 8, 12, 16, 20 or 24 hours) at a temperature of approximately 30° C. (for example, approximately 22, 24, 26, 28, 30, 32, 34, 36 or 38° C.).

Optionally, fermentation-improving cofactors such as alpha-ketoglutarate, manganese and/or magnesium salts may also be added prior to the fermentation.

In one embodiment, the method of the invention produces a flavoured milk product with a concentration of 2,3,5,6-tetramethyl pyrazine of at least 10 parts-per-million (ppm), preferably at least about 20 ppm, more preferably at least about 30 ppm, most preferably at least about 40 ppm, and particularly at least about 50 ppm. In one embodiment, the 2,3,5,6-tetramethyl pyrazine has a concentration of about 10 to about 1000 ppm, preferably about 20 to about 500 ppm, more preferably about 30 to about 250 ppm, most preferably about 40 to about 150 ppm, and particularly about 50 to about 100 ppm.

In one embodiment, the flavoured milk product comprises 5-methyl-2-phenyl-2Z-hexenal and/or 2-phenylbutenal; preferably wherein the 5-methyl-2-phenyl-2Z-hexenal and/or 2-phenylbutenal are produced in the fermented milk source.

In one embodiment, wherein the 5-methyl-2-phenyl-2Z-hexenal and/or 2-phenylbutenal provide further and/or enhanced chocolate flavours and/or aromas to the flavoured milk product.

In one embodiment, the amino acid supplemented milk is fermented with at least the bacterium deposited at the CNCM under the following accession number: CNCM I-1962. The present inventors have found that the use of this bacterium advantageously increases the production of 5-methyl-2-phenyl-2Z-hexenal (also known as cocal), which is another compound associated with a chocolate flavour and/or aroma.

5-methyl-2-phenyl-2Z-hexenal is produced by the reaction of phenyl acetaldehyde with 3-methylbutenal, which is catalysed by the enzyme aldolase. This enzyme is endogenous in the bacterium CNCM I-1962. Phenyl acetaldehyde is derived from the amino acid L-phenylalanine and/or L-leucine, which undergoes a transamidation reaction to form phenylpyruvic acid, which is in turn decarboxylated to form phenyl acetaldehyde. 3-methylbutenal is derived from the amino acid L-leucine via a transamidation reaction. These reactions are catalysed by enzymes present in the at least one microorganism.

Thus, in one embodiment, the flavoured milk product further comprises 5-methyl-2-phenyl-2Z-hexenal. In one embodiment, the 5-methyl-2-phenyl-2Z-hexenal is present at a concentration of: greater than about 1 ppm, about 1-1000 ppm, about 5-500 ppm, about 10-200 ppm, about 10-100 ppm, about 20 to about 100 ppm or about 20-50 ppm.

In one embodiment, the amino acid supplemented milk is fermented using the bacteria deposited at the CNCM under the following accession numbers: CNCM I-1962 and CNCM I-2116, either together or sequentially (in either order). In one embodiment the amino acid supplemented milk is fermented using bacterium CNCM I-2116 and CNCM I-1962 together or first with the bacterium CNCM I-1962 and then with the bacterium CNCM I-2116. The present inventors have found that the use of these bacteria together or sequentially advantageously increases the production of 2-phenylbutenal, which is a further compound associated with a chocolate flavour and/or aroma.

2-phenylbutenal is formed from the reaction of phenyl acetaldehyde and acetaldehyde, also catalysed by the enzyme aldolase. The bacterium CNCM I-2116 has been found by the inventors to provide efficient formation of the necessary acetaldehyde, thus increasing the production of 2-phenylbutenal. Phenyl acetaldehyde is derived from L-phenylalanine as noted above; acetaldehyde may be derived via a number of different chemical pathways, including from pyruvate.

Thus, in one embodiment, the flavoured milk product further comprises 2-phenylbutenal. In one embodiment, the 2-phenylbutenal is present at a concentration of: greater than about 5 ppm, about 10-1000 ppm, about 20-500 ppm, about 20-200 ppm, or about 50-200 ppm.

Following the step of increasing the pH of the fermented milk source using ammonia, the flavoured milk product may be dried and/or homogenised.

The present inventors have found that the production of 2,3,5,6-tetramethyl pyrazine is particularly efficient when the fermented milk source is heated following the use of ammonia to increase the pH.

Thus, in a preferred embodiment, the method of the invention further comprises subjecting the fermented milk source to a heat treatment.

The heat treatment step is carried out either at the same time as step (c) of the method of the invention, or following step (c). Where the heat treatment step is carried out following step (c) of the method of the invention, said heat treatment step may form step (d) of the method.

The use of a heat treatment step can increase the production of 2,3,5,6-tetramethyl pyrazine at least about 2-fold, 4-fold, 5-fold, or 10-fold.

In one embodiment, the heat treatment comprises spray drying. In spray drying, a liquid (such as a milk source) is sprayed through a small nozzle into a heated drying gas. This produces a dried powder or particles which can subsequently be collected.

In one embodiment, the heat treatment comprises at least one of Ultra High Temperature (UHT) processing, spray drying, roller drying, vacuum belt drying, continuous drying or batch drying, preferably UHT process or spray drying.

In a UHT processing treatment, a food product (such as one derived from a milk source) is subjected to high temperatures of greater than about 130° C. (preferably about 135-155° C., more preferably about 140-150° C., and most preferably about 145-148° C.). The UHT treatment may be carried out over a period of less than about 10 seconds (preferably about 1-7 seconds, particularly about 2-6 seconds, more preferably about 2-5 seconds and most preferably about 2-4 seconds).

Thus, in a preferred embodiment, the invention provides (A) a method for producing a flavoured milk product, the method comprising the steps of:

(a) providing a milk source;

(b) fermenting the milk source using at least one microorganism, to produce a fermented milk source having an acidic pH;

(c) increasing the pH of the fermented milk source using ammonia; and

(d) subjecting the fermented milk source to a heat treatment;

wherein the ammonia reacts with the fermented milk source to produce 2,3,5,6-tetramethyl pyrazine.

In a further preferred embodiment, the invention provides (B) a method for producing a flavoured milk product, the method comprising the steps of:

(a) providing a milk source;

(b) fermenting the milk source using at least one microorganism, to produce a fermented milk source having an acidic pH;

(c) increasing the pH of the fermented milk source using ammonia; and

(d) subjecting the fermented milk source to a heat treatment comprising spray drying;

wherein the ammonia reacts with the fermented milk source to produce 2,3,5,6-tetramethyl pyrazine.

Thus, in yet a further preferred embodiment, the invention provides (C) a method for producing a flavoured milk product, the method comprising the steps of:

(a) providing a milk source;

(b) fermenting the milk source using at least one microorganism, to produce a fermented milk source having an acidic pH;

(c) increasing the pH of the fermented milk source using ammonia; and

(d) subjecting the fermented milk source to a heat treatment comprising UHT processing;

wherein the ammonia reacts with the fermented milk source to produce 2,3,5,6-tetramethyl pyrazine.

Thus, in a preferred embodiment, the invention provides a method (D) for producing a flavoured milk product, the method comprising the steps of:

(a) providing an amino acid supplemented milk source;

(b) fermenting the amino acid supplemented milk source using at least one microorganism, to produce a fermented milk source having an acidic pH;

(c) increasing the pH of the fermented milk source using ammonia; and

(d) subjecting the fermented milk source to a heat treatment;

wherein the ammonia reacts with the fermented milk source to produce 2,3,5,6-tetramethyl pyrazine.

Thus, in a further preferred embodiment, the invention provides (E) a method for producing a flavoured milk product, the method comprising the steps of:

(a) providing an amino acid supplemented a milk source;

(b) fermenting the amino acid supplemented milk source using at least one microorganism, to produce a fermented milk source having an acidic pH;

(c) increasing the pH of the fermented milk source using ammonia; and

(d) subjecting the fermented milk source to a heat treatment comprising spray drying;

wherein the ammonia reacts with the fermented milk source to produce 2,3,5,6-tetramethyl pyrazine.

Thus, in yet a further preferred embodiment, the invention provides (F) a method for producing a flavoured milk product, the method comprising the steps of:

(a) providing an amino acid supplemented a milk source;

(b) fermenting the amino acid supplemented milk source using at least one microorganism, to produce a fermented milk source having an acidic pH;

(c) increasing the pH of the fermented milk source using ammonia; and

(d) subjecting the fermented milk source to a heat treatment comprising UHT processing;

wherein the ammonia reacts with the fermented milk source to produce 2,3,5,6-tetramethyl pyrazine.

In any of the methods (A)-(F) above the at least one microorganism in step (b) is a lactic acid bacterium as defined in any embodiment disclosed herein. Preferably the lactic acid bacterium is Lactococcus lactis, more preferably Lactococcus lactis subsp. Lactis, and most preferably Lactococcus lactis subsp. Lactis biovar. Diacetylactis, and most preferably a lactic acid bacterium selected from the bacteria deposited at the Collection Nationale de Cultures de Microorganismes under the following accession numbers: CNCM I-1962, CNCM I-2116, CNCM I-4404 and CNCM I-4405; preferably CNCM I-1962, CNCM I-4404 and CNCM I-4405. Particularly preferred are lactic acid bacterium selected from CNCM I-1962, or a combination of CNCM I-1962 and CNCM I-2116 together or sequentially. These bacteria advantageously increases the production of 5-methyl-2-phenyl-2Z-hexenal and/or 2-phenylbutenal, particularly when the milk source in step (a) is an amino acid supplemented milk source [i.e. embodiments (D), (E) and (F)].

In any of the methods (A)-(F), the ammonia may be added in the form of ammonium hydroxide, or an ammonium salt. Ammonia may also be generated in situ by the addition of a urease enzyme as described above. When a urease enzyme is used to generate ammonia in situ, the pH may be adjusted to about pH 6.5-7.5 by the use of a base (e.g. potassium hydroxide or sodium hydroxide), in order to provide optimum conditions for urease activity.

In any of the methods (D-F), the amino acid supplemented milk source is preferably prepared by adding at least one protease to a milk source, particularly to increase the amount of free L-phenylalanine, and optionally to additionally increase the amount of L-leucine, L-isoleucine and L-valine. The protease may be derived from a microorganism, for example Aspergillus oryzae, Aspergillus sojae, Rhizophus oryzae, Bacillus subtilis, L. helveticus, S. thermophiles, L. plantarum, or L. lactis subspecies, or from plants such as Ananas comosus. Preferably the protease is derived from Aspergillus oryzae, more preferably the strain ATCC 42149/RIB40 (yellow koji mould)

As well as supplementation with amino acids, the milk product may be further supplemented with one or more additional compounds prior to the fermentation step, to provide additional substrates which may be converted by the at least one microorganism directly or indirectly into further flavour- and/or aroma-associated compounds.

In one embodiment, the milk source is supplemented with citrate (for example in the form of trisodium citrate) prior to the fermentation step. The citrate (e.g. trisodium citrate) may be added to the milk source in an amount of 0.1 to 5 wt. %, preferably 0.01 to 2 wt. %, more preferably 0.03 to 1.0 wt. %, most preferably 0.05 to 0.3 wt. %

Lipase can be added to the milk source to produce further desired flavours in the milk products. Lipase hydrolyses fats in the milk source to form for example di-glycerides, monoglycerides and free fatty acids or any mixtures thereof. The di-glycerides, monoglycerides and free fatty acids impart a cream-like flavour to the fermented milk product.

Lactase can also be added to the milk source or the mixture to produce further desired flavours in the milk products. Lactase hydrolyses the disaccharide lactose in the milk source into galactose and glucose. Glucose and galactose can be used as flavour precursors for caramel-like and sweetened condensed milk-like flavour formation.

In another aspect, the invention provides a flavoured milk product obtainable by the method of the invention, as described herein.

A fermented milk product produced according to the method of the invention can have applications in food products and during the manufacture of food products. For example, a flavoured milk product, as provided by the present invention, can be used to impart a chocolate flavour and/or aroma to another food or beverage.

A food product containing a flavoured milk product may be prepared by a process comprising the steps of: (a) preparing a flavoured milk product by a process as described above, and (b) adding the flavoured milk product to a food to form a food product.

The flavoured milk product of the invention may be used to impart a flavour to a food product by a process comprising the steps of: (a) preparing a flavoured milk product by a process as described above, and (b) adding the flavoured milk product to a food product.

A flavoured milk product or food product as described herein may be in the form of a liquid or a solid, preferably a solid, and more preferably a powder.

It will be appreciated that the fermented milk product prepared by the present invention enables the generation of a chocolate flavour without the need to add cocoa or chocolate products or flavouring. In particular a chocolate flavour in the milk products of the present invention can be attributed to the process of the present invention, whereby ammonia is reacted (as a solution of ammonia, a solution of an ammonia source such as an ammonium salt, or generated in situ in the fermented milk source by the addition of urease enzyme) with a fermented milk source to produce 2,3,5,6-tetramethyl pyrazine. The production of 2,3,5,6-tetramethyl pyrazine is further enhanced by heat treating the fermented milk source in the presence of ammonia. The heat treatment can be conducted before the addition of ammonia, or afterwards. For example, the heat treatment can be conducted afterwards by a spray drying step, or by UHT treatment as described herein. Preferably, when a urease is used, the heat treatment is conducted after allowing the mixture comprising urease and fermented milk source to incubate for an appropriate time, preferably under the conditions described above.

The food product is preferably selected from dairy desserts, yogurts, dairy-based nutritional compositions or ice-creams. Dairy-based nutritional composition encompass for example growing-up milks, dairy-based nutritional compositions with thickened texture and cereal milk drinks. The food product may be chilled, shelf stable or frozen. In an embodiment it is chilled or shelf-stable.

Those skilled in the art will understand that they can freely combine all features of the present invention disclosed herein. In particular, features described for the product of the present invention may be combined with the process of the present invention and vice versa.

Furthermore, features described for different embodiments of the present invention may be combined. Where known equivalents exist to specific features, such equivalents are incorporated as if specifically referred to in this specification. Further advantages and features of the present invention are apparent from the Figures and non-limiting Examples.

### EXAMPLES

Various preferred features and embodiments of the present invention will now be described by way of non-limiting examples.

### Example 1

Full cream milk powder was reconstituted to a concentration of 35% to provide a milk source. Amino acids L-leucine, L-isoleucine, L-valine and L-phenylalanine were added to the milk source at the concentrations shown in Table 1 below.

The amino acid supplemented milk source was preheated to 80° C. followed by a UHT treatment comprising heating the amino acid supplemented milk source to a temperature of 140° C. for 4 seconds.

Following the UHT treatment, a culture of the bacterium CNCM I-1962 was added to the amino acid supplemented milk source at a concentration of 1%, as shown in Table 1 below.

TABLE 1

| | Concentration at start of | Composition % TS |
|---|---|---|
| Ingredient | fermentation % | (total solids) |
| Full cream milk powder | 30 | 99.2 |
| CNCM I-1962 culture | 1 | 0.3 |
| L-leucine | 0.03 | 0.075 |
| L-isoleucine | 0.03 | 0.075 |
| L-valine | 0.02 | 0.075 |
| L-phenylalanine | 0.03 | 0.075 |

Fermentation took place at 30° C. under aerobic conditions for approximately 10 hours.

Urease enzyme was added. The pH was adjusted to approximately 7 (e.g. using a base, such as potassium hydroxide or sodium hydroxide) in order to obtain a pH for good activity of the urease enzyme.

The fermented milk source was cooled and spray dried to form a powder.

Chemical analysis confirmed the presence of 2,3,5,6-tetramethyl pyrazine.

Sensory evaluation by a trained panel revealed that the obtained fermented milk product exhibited a chocolate flavour.

### Example 2

Amino acid supplemented milk was fermented with the lactic acid bacterium CNCM I-4404. Following fermentation, the milk was neutralised using ammonia before being dried.

It was found that the longer drying times led to a significant increase in 2,3,5,6-tetramethyl pyrazine production.

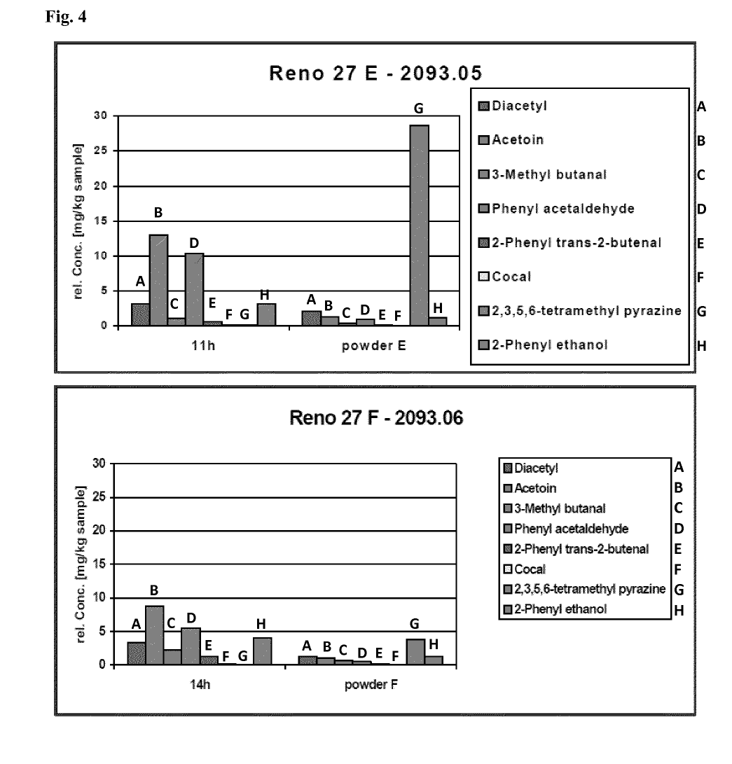

FIG. 4 shows two example powders. Powder 2093.05 underwent longer drying than powder 2093.06, and has a significantly higher concentration of 2,3,5,6-tetramethyl pyrazine (shown in the second to last bar on the right hand side of the graph).

Sensory evaluation by trained panel also revealed that the powder 2093.05 was characterized by a significantly stronger chocolate flavour.

### Example 3

The effect of the basic substance used to neutralise the fermented milk on 2,3,5,6-tetramethyl pyrazine was determined.

2,3,5,6-tetramethyl pyrazine formation when the fermented milk was neutralised with ammonia (in the form of ammonium hydroxide, NH₄OH) was compared to 2,3,5,6-tetramethyl pyrazine formation when the fermented milk was neutralised with potassium hydroxide (KOH). In both cases, neutralisation was followed by spray drying.

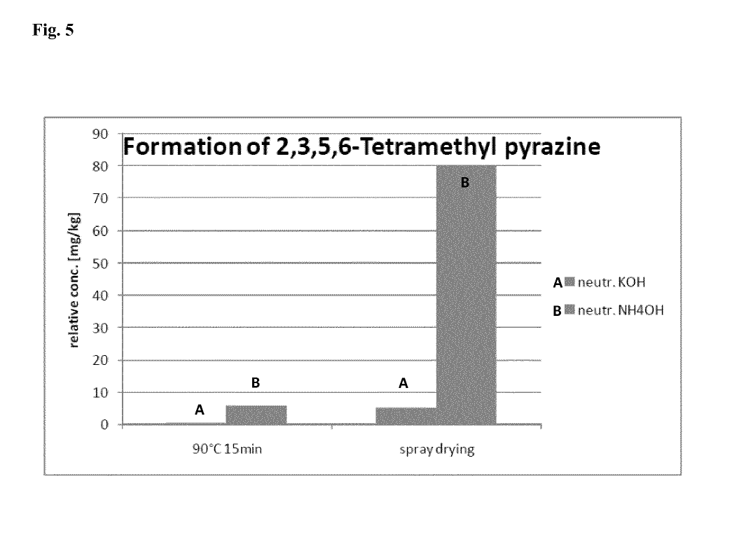

FIG. 5 shows that formation of 2,3,5,6-tetramethyl pyrazine was significantly increased when neutralisation was carried out using ammonia as compared to potassium hydroxide.

### Example 4

Full cream milk powder was reconstituted to a concentration of 35% to provide a milk source. Amino acids L-leucine, L-isoleucine, L-valine and L-phenylalanine were added to the milk source at the concentrations shown in Table 1 below.

Then, a culture of the bacteria CNCM I-4404 and CNCM I-1962 was added to the amino acid supplemented milk source at a concentration of 1%, as shown in Table 1 below.

TABLE 1

| | Concentration at start of | Composition % TS |
|---|---|---|
| Ingredient | fermentation % | (total solids) |
| Full cream milk powder | 30 | 99.2 |
| CNCM I-1962 culture | 0.5 | 0.15 |
| CNCM I-4404 | 0.5 | 0.15 |
| L-leucine | 0.03 | 0.075 |
| L-isoleucine | 0.03 | 0.075 |
| L-valine | 0.02 | 0.075 |
| L-phenylalanine | 0.03 | 0.075 |

The amino acid supplemented milk source was then fermentated at 30° C. under aerobic conditions for approximately 12 hours.

The pH was then adjusted to 6.7 using ammonia.

The fermented milk source was cooled and spray dried to form a powder.

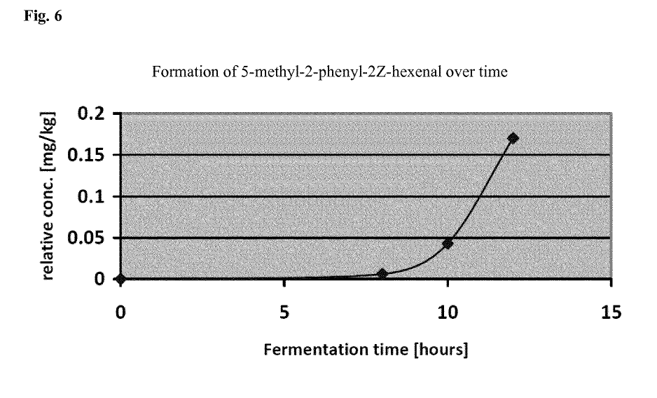

The concentration of 5-methyl-2-phenyl-2Z-hexenal was measured at different points in time during fermentation. The results are reported in FIG. 6, which shows that 5-methyl-2-phenyl-2Z-hexenal started to be formed after 8 hours of fermentation.

Sensory evaluation by a trained panel of the fermented milk product obtained in this example revealed that it was characterized by a chocolate, malt and honey flavour.

1. A process for producing a flavored milk product comprising the steps of:
(a) providing a milk source;
(b) fermenting the milk source using at least one microorganism, to produce a fermented milk source having an acidic pH; and
(c) increasing the pH of the fermented milk source using ammonia, wherein the ammonia reacts with the fermented milk source to produce 2,3,5,6-tetramethyl pyrazine, thereby producing a flavored milk product.

2. A process according to claim 1, wherein the ammonia in step (c) is produced by adding urease to the fermented milk source, and/or wherein step (c) comprises adding ammonia or an ammonium salt to the fermented milk source.

3. A process according to claim 1, wherein the pH in step (c) is increased to about 6 to about 8.0.

4. A process according to claim 1, wherein the step (c) further comprises subjecting the fermented milk source to heat treatment.

5. A process according to claim 1, wherein the at least one microorganism is a lactic acid bacterium (Lactobacillales).

6. A process according to claim 1, wherein the at least one microorganism is a Lactococcus bacterium.

7. A process according to claim 5, wherein the microorganism is selected from the bacteria deposited at the Collection Nationale de Cultures de Microorganismes under the following accession numbers: CNCM I-1962, CNCM I-2116, CNCM I-4404 and CNCM I-4405; preferably CNCM I-1962, CNCM I-4404 and CNCM I-4405.

8. A process according to claim 1, wherein the 2,3,5,6-tetramethyl pyrazine is present at a concentration of at least about 1 parts-per-million (ppm).

9. A process according to claim 1, wherein the milk source in step (a) is an amino acid supplemented milk source.

10. A process according to claim 1, wherein the milk source in step (a) is an amino acid supplemented milk source comprising one or more amino acids selected from the group consisting of: L-phenylalanine, L-leucine, L-isoleucine and L-valine.

11. A process according to any of claim 9 or 10, wherein the amino acid supplemented milk source is fermented using the bacterium deposited at the Collection Nationale de Cultures de Microorganismes under the accession number CNCM I-1962.

12. A process according to claim 11, wherein the flavoured milk product further comprises 5-methyl-2-phenyl-2Z-hexenal.

13. A process according to claim 9, wherein the amino acid supplemented milk source is fermented using the bacteria deposited at the Institut Pasteur Collection Nationale de Cultures de Microorganismes under the following accession numbers: CNCM I-1962 and CNCM I-2116, together or sequentially.

14. A process according to claim 13, wherein the flavoured milk product further comprises 2-phenylbutenal.

15. A process according to claim 9, wherein the amino acid supplemented milk source in step (a) is prepared by hydrolysis of milk proteins in a milk source.

16. (canceled)

17. A process for preparing a food product containing a flavored milk product, comprising the steps of:
(a) preparing a flavoured milk product by a process comprising the steps of: providing a milk source; fermenting the milk source using at least one microorganism, to produce a fermented milk source having an acidic pH; and increasing the pH of the fermented milk source using ammonia, wherein the ammonia reacts with the fermented milk source to produce 2,3,5,6-tetramethyl pyrazine, thereby producing a flavored milk product, and
(b) adding the flavoured milk product to a food to form a food product.

18. (canceled)

19. A process according to claim 17, wherein the milk product has a chocolate flavour.

20. A flavoured milk product comprising 2,3,5,6-tetramethyl pyrazine at a concentration of at least about 10 parts-per-million (ppm).

21. A flavoured milk product according to claim 20 further comprising 5-methyl-2-phenyl-2Z-hexenal.

22-26. (canceled)
